(19) United States
(12) Patent Application Publication
(10) Pub. No.: US 2024/0173126 A1
(43) Pub. Date: May 30, 2024

(54) TEXTILES AND MEDICAL DEVICES USING THE SAME

(71) Applicant: EDWARDS LIFESCIENCES CORPORATION, Irvine, CA (US)

(72) Inventors: Sandip Vasant Pawar, Irvine, CA (US); Delfin Rafael Ruiz, Sun Valley, CA (US)

(21) Appl. No.: 18/431,739

(22) Filed: Feb. 2, 2024

Related U.S. Application Data
(63) Continuation of application No. PCT/US2022/042604, filed on Sep. 6, 2022.
(60) Provisional application No. 63/241,330, filed on Sep. 7, 2021.

(51) Int. Cl. A61F 2/24; A61F 2/07; D02G 3/04; D02G 3/32; D02G 3/36; D02G 3/38; D03D 1/00; D03D 13/00; D03D 15/283; D03D 15/47; D03D 15/56; D04B 1/18; D04B 1/22; D04C 1/02; D04C 1/06

(52) U.S. Cl. CPC A61F 2/2418; D02G 3/045; D02G 3/32; D02G 3/36; D02G 3/38; D03D 1/00; D03D 13/004; D03D 15/283; D03D 15/47; D03D 15/56; D04B 1/18; D04B 1/22; D04C 1/02; D04C 1/06; A61F 2002/077; D10B 2331/04; D10B 2331/10; D10B 2401/061; D10B 2509/00

(57) ABSTRACT

Disclosed herein is a textile comprising: a) a first yarn comprising one or more filaments comprising a first polymer; b) one or more additional yarns comprising a composition and/or properties different from the first yarn; wherein the first yarn exhibits an elasticity higher than an elasticity of the one or more additional yarns the textile exhibits an ultimate elongation of about 50% to about 700% and is shape-adapting and biocompatible, and wherein the textile is configured to be inserted and/or implanted in a subject's body. Also described herein are medical devices having the disclosed textiles. In addition, disclosed herein are methods of making the disclosed textiles.

### CROSS-REFERENCE TO RELATED APPLICATIONS

This application is a continuation of PCT patent application no. PCT/US2022/042604, filed Sep. 6, 2022, which claims the benefit of U.S. Provisional Application No. 63/241,330, filed Sep. 7, 2021, the content of which is incorporated herein by reference in its entirety.

### FIELD

The present application concerns aspects of textile materials comprising yarns having different elasticities. The present application also concerns aspects of implantable medical devices comprising such textile materials.

### BACKGROUND

Many medical devices often incorporate textiles and fabrics that can fulfill various functions, depending on the nature and type of the medical device. These textiles can be used in various applications ranging from use in delivery devices, sutures, wound dressings, implantation valves, and skin grafts to arterial grafts, among many others.

For example, implantable bioprosthetic heart valves and stent grafts often include a layer of fabric, such as an outer covering or inner or outer fabric skirt, which is attached to a metal frame or stent. In the case of bioprosthetic heart valves, the fabric skirt can be used to anchor the valve leaflets to the stent. The fabric skirt can also function to protect the delicate valve leaflets from damage that may otherwise occur from direct contact between the valve leaflets and the stent when the heart valve is radially compressed for delivery through a patient's vasculature.

The fabric present in such an exemplary device needs to possess various properties, such as, for example, a desired level of stretchability to allow the textile to conform to the changes in the valve shape and/or its dimensions when it expands from the collapsed configuration. Similarly, the textiles and fabrics need to be shape-adaptive to conform to the body's natural anatomy and allow prevention of a blood leak upon insertion and the like.

While stretchable textiles and fabrics comprising various elastomeric fibers are very popular in making comfortable clothing, shapewear, exercise wear, and such, these conventional textiles are not easily adapted for medical usage when the textile is in contact with a subject's body and/or body liquids, as such textile need to possess additional properties in order to be compatible with the subject's body and not harmful in any way.

These needs and others are at least partially satisfied by the present disclosure.

### SUMMARY

Some of the aspects of the present disclosure relate to textiles. Some aspects relate to a textile comprising: a) a first yarn comprising one or more filaments comprising a first polymer; b) one or more additional yarns comprising a composition and/or properties different from the first yarn; wherein the first yarn exhibits an elasticity higher than an elasticity of the one or more additional yarns the textile exhibits an ultimate elongation of about 50% to about 700% and is shape-adapting and biocompatible, and wherein the textile is configured to be inserted and/or implanted in a subject's body.

In yet other aspects, the first polymer can comprise a thermoplastic polyurethane (TPU), a polyurethane (PU), a thermoplastic elastomer (TPE), and an elastomeric polyolefin, polybutylene terephthalate (PBT), silicone-based elastomer, or a combination thereof.

In some aspects, the first yarn can be a monofilament. While in other aspects, the first yarn can be a multifilament.

In yet further aspects, the one or more additional yarns can comprise a fully-drawn yarn, spin-drawn yarn, low- or not-twisted yarn, twisted yarn, a flat yarn, a textured yarn, a high (HS) stretch textured yarn, a high-bulk textured yarn, a high shrinkage yarn, or any combination thereof.

In still further aspects, the one or more additional yarns comprise a polyolefin, a polyester, a polyurethane, a thermoplastic polyurethane, a polyamide, or a combination thereof.

In addition, or in alternative also disclosed are aspects wherein the textile further comprises a second yarn comprising one or more filaments comprising a second polymer. In such aspects, the second yarn is different from the first yarn. In yet further aspects, the second yarn can cover the first yarn to form a multi-component yarn such that the first yarn forms a core and the second yarn forms a sheath. In yet still further aspects, the first polymer has higher elasticity than the second polymer. In yet still further aspects, the second polymer can comprise a polyester, a nylon, a polyolefin, or a combination thereof.

In still further aspects, the textiles disclosed herein can have a knitted, woven, or a braided construction.

Also disclosed herein are articles comprising any of the disclosed herein textiles.

In certain aspects, disclosed is an implantable prosthetic valve comprising: an annular frame having an inflow end, an outflow end, and a longitudinal axis, wherein the annular frame is configured to expand and collapse; and a sealing element secured to the frame, wherein the sealing element comprises a textile comprising: a) a first yarn comprising one or more filaments comprising a first polymer; b) one or more additional yarns comprising a composition and/or properties different from the first yarn; wherein the first yarn exhibits an elasticity higher than an elasticity of the one or more additional yarns the textile exhibits an ultimate elongation of about 50% to about 700% and is shape-adapting and biocompatible, and wherein the textile is configured to be inserted and/or implanted in a subject's body.

In still further aspects, disclosed herein are medical devices comprising any of the disclosed herein textiles.

Also, in some aspects, disclosed herein are methods comprising forming a textile comprising: a) a first yarn comprising one or more filaments comprising a first polymer; b) one or more additional yarns that have a composition and/or properties different from the first yarn; and c) optionally a second yarn comprising one or more filaments comprising a second polymer, wherein if the second yarn is present, the second yarn covers the first yarn to form a multi-component yarn such that the first yarn forms a core and the second yarn forms a sheath, wherein the first yarn exhibits an elasticity higher than an elasticity of the one or more additional yarns and of the second yarn if present; and wherein the textile exhibits an ultimate elongation of about 50% to about 700% and is shape-adapting and biocompatible, is configured to be inserted and/or implanted in a subject's body.

In certain aspects, the step of forming comprising weaving, knitting, braiding, or any combinations thereof.

Additional aspects of the disclosure will be set forth, in part, in the detailed description, figures, and claims which follow, and in part will be derived from the detailed description or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIGS. 1A-1B show an exemplary monofilament multi-component yarn in one aspect: FIG. 1A depicts a single covered multi-component yarn where a monofilament first yarn is covered with a monofilament second yarn, and FIG. 1B depicts a double-covered multi-component yarn where a monofilament first yarn is covered with two layers of a monofilament second yarn.

FIGS. 2A-2B show an exemplary multi-component yarn in one aspect: FIG. 2A depicts a single covered multi-component yarn where a monofilament first yarn is covered with a multifilament second yarn and FIG. 2B depicts a double-covered multi-component yarn where a monofilament first yarn is covered with two layers of a multifilament second yarn.

FIGS. 3A-3B show an exemplary multi-component yarn in one aspect: FIG. 3A depicts a single covered multi-component yarn where a multifilament first yarn is covered with a multifilament second yarn and FIG. 3B depicts a double-covered multi-component yarn where a multifilament first yarn is covered with two layers of a multifilament second yarn.

FIG. 4 shows an exemplary woven pattern for an exemplary textile in one aspect.

FIGS. 5A-5D show an exemplary knitted pattern for an exemplary textile in one aspect: FIG. 5A depicts an exemplary simple single jersey knit construction; FIG. 5B depicts an exemplary single jersey knit with knit-miss pattern construction (left a photograph of the constructions; right-a schematic of the construction); FIG. 5C depicts exemplary single jersey knit with knit-tuck pattern constructions; FIG. 5D depicts an exemplary plaited knit-tuck textile construction in one aspect.

FIG. 6 shows a perspective view of an exemplary implantable medical device, such as a prosthetic heart valve, including an exemplary aspect of an exemplary textile used as a paravalvular leakage seal.

FIG. 7 shows a perspective view illustrating an exemplary implantable medical device, such as a prosthetic heart valve, including another aspect of a paravalvular leakage seal, including an exemplary textile used as a paravalvular leakage seal.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

As used in this application and in the claims, the singular forms “a,” “an,” and “the” include the plural forms unless the context clearly dictates otherwise. Thus, for example, reference to a “yarn” includes aspects having two or more such yarns unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example,” “exemplary,” and “such as,” and grammatical equivalences thereof, the phrase “and without limitation” is understood to follow unless explicitly stated otherwise.

Ranges can be expressed herein as from “about” one particular value and/or to “about” another particular value. When such a range is expressed, another aspect includes from one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur and that the description includes instances where said event or circumstance occurs and instances where it does not.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s). It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially identical” or “substantially similar” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the terms “substantially identical reference composition,” or “substantially identical reference article,” or “substantially identical reference method” refer to a reference composition or article or a method comprising substantially identical components or steps in the absence of an inventive component or a step. In another exemplary aspect, the term “substantially,” in, for example, the context “substantially identical reference composition,” refers to a reference composition comprising substantially identical components and wherein an inventive component is substituted with a common in the art component.

As used herein, the term “and/or” includes any and all combinations of one or more of the associated listed items.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

The term “fiber” as used herein includes fibers of extreme or indefinite length (i.e., filaments) and fibers of short length (i.e., staple fibers).

As used herein, the term “polymers” refers to molecules containing a plurality of one or more monomeric units (e.g., from 2 to 5 to 10 to 25 to 50 to 100 to 250 to 500 to 1000 or more copies). The polymers disclosed herein can have various architectures, for example, and without limitations, cyclic, linear, and branched architectures. Branched architectures include star-shaped architectures (e.g., configurations in which three or more chains emanate from a single branch point), comb architectures (e.g., architectures having a main chain and a plurality of side chains), and dendritic architectures (e.g., arborescent and hyperbranched polymers), among others.

As used herein, the term “homopolymers” are polymers that contain multiple copies of a single monomeric unit. The term “copolymers” refers to polymers that contain multiple copies of at least two dissimilar monomeric units, examples of which include random, gradient, periodic (e.g., alternating), and block copolymers. As used herein, the term “block copolymers” refers to copolymers that contain two or more polymer blocks that differ in composition, for instance, a monomeric that is found in one polymer block may not be found in another polymer block. As used herein, the term “polymer block” is a grouping of monomeric units (e.g., 2 to 5 to 10 to 25 to 50 to 100 to 250 to 500 to 1000 or more units). Blocks can be branched or unbranched. Blocks can contain a single type of monomeric unit (also referred to herein as “homopolymeric blocks”), or they can contain multiple types of monomeric units (also referred to herein as “copolymeric blocks”) which can be provided, for example, in a random, gradient, or periodic (e.g., alternating) distribution

As used herein, the term “polyester” refers to a category of polymers that contain the ester functional group in their main chain. Polyesters disclosed herein include naturally occurring chemicals, such as in the cutin of plant cuticles, as well as synthetics produced through step-growth polymerization. In certain examples, the polyesters comprise polyethylene terephthalate (PET) homopolymer and copolymers, polypropylene terephthalate (PPT) homopolymer and copolymers and polybutylene terephthalate (PBT) homopolymer and copolymers, and the like, including those that contain comonomers such as cyclohexane dimethanol, cyclohexane dicarboxylic acid, isophthalic acid, and the like.

The term “polyamide,” as utilized herein, is defined to be any long-chain polymer in which the linking functional groups are amide (—CO—NH—) linkages. The term polyamide is further defined to include copolymers, terpolymers, and the like, as well as homopolymers, and also includes blends of two or more polyamides. In some aspects, the plurality of polyamide fibers comprise one or more of nylon 6, nylon 66, nylon 10, nylon 612, nylon 12, nylon 11, or any combination thereof. In other aspects, the plurality of polyamide fibers comprise nylon 6 or nylon 66. In yet other aspects, the plurality of polyamide fibers are nylon 6. In a yet further aspect, the plurality of polyamide fibers are nylon 66.

As defined herein, the term “polyolefin” refers to any class of polymers produced from a simple olefin (also called an alkene with the general formula CₙH₂ₙ) as a monomer. In some aspects, the polyolefins include but are not limited to polyethylene, polypropylene, both homopolymer and copolymers, poly(I-butene), poly(3-methyl-1-butene), poly(4-methyl-1-pentene) and the like, as well as combinations or mixtures of two or more of the foregoing.

As defined herein, the term “polyurethane” refers to any class of polymers composed of a chain of organic units joined by carbamate (urethane, R₁—O—CO—NR₂-R₃, wherein R1, R2, and R3 are the same or different) links.

As defined herein, the term “polyether” refers to any class of polymers composed of a chain of organic units joined by an ether group.

As defined herein, the term “polyurea” refers to any class of polymers where alternative monomer units of isocyanates and amines react with each other to form urea linkages.

As defined herein, the term “polystyrene” refers to any class of synthetic polymers produced from a simple styrene as a monomer. It is understood that the term “polystyrene” includes both atactic and syndiotactic polystyrenes. In some specific aspects, described are also co-polystyrenes, including high-impact polystyrenes (HIPS), acrylonitrile butadiene styrene (ABS), or copolymer of styrene with acrylonitrile (SAN), or copolymer of styrene with maleic acid (SMA).

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### Textile

Disclosed herein is a textile comprising: a) a first yarn comprising one or more filaments comprising a first polymer; b) one or more additional yarns comprising a composition and/or properties different from the first yarn. In still further aspects, the first yarn present in the disclosed herein textile has elasticity higher than an elasticity of the one or more additional yarns. In still further aspects, the textile can exhibit an ultimate elongation of about 50% to about 700%, including exemplary values of about 100%, about 150%, about 200%, about 250%, about 300%, about 350%, about 400%, about 450%, about 500%, about 550%, about 600%, and about 650%.

In still further aspects, the textile is shape-adapting and biocompatible. While in still further aspects, the textile is configured to be inserted and/or implanted in a subject's body.

It is understood that the first yarn and the one or more additional yarns are also biocompatible. In still further aspects, the first yarn and the one or more additional yarns are medical-grade yarns.

In still further aspects, the first yarn is an elastomeric yarn. In such aspects, the first polymer comprises an elastomer. In still further aspects, the first polymer can comprise a thermoplastic polyurethane (TPU), a polyurethane (PU), a thermoplastic elastomer (TPE), and an elastomeric polyolefin, polybutylene terephthalate (PBT), silicone-based elastomer, or a combination thereof. In yet other aspects, the first polymer is a thermoplastic polyurethane (TPU). While in other aspects, the first polymer a polyurethane (PU). In yet further aspects, the first polymer is a thermoplastic elastomer (TPE). While in still further aspects, the first polymer is an elastomeric polyolefin. In still further and unlimiting aspects, the first polymer is polybutylene terephthalate (PBT). While in still further aspects, the first polymer is a silicone-based elastomer.

In still further aspects, the first polymer can have a durometer from about 75 A to about 75 D, including exemplary values of about 80 A, about 85 A, about 90 A, about 95 A, about 100 A, about 20 D, about 25 D, about 30 D, about 35 D, about 40 D, about 45 D, about 50 D, about 55 D, about 60 D, about 65 D, and about 70 D. In yet other aspects, the first polymer can have a durometer value between any two foregoing values.

In still further aspects, the first polymer can have an ultimate tensile strength between about 4500 psi to about 8000 psi, including exemplary values of about 5000 psi, about 5500 psi, about 6000 psi, about 6500 psi, about 7000 psi, and about 7500 psi. In yet other aspects, the first polymer can have an ultimate tensile strength between any two foregoing values.

In still further aspects, the first polymer can have an ultimate elongation between about 50% to about 700%, including exemplary values of about 100%, about 150%, about 200%, about 250%, about 300%, about 350%, about 400%, about 450%, about 500%, about 550%, about 600%, and about 650%. In yet other aspects, the first polymer can have an ultimate elongation value between any two foregoing values.

In certain aspects, the first yarn can be a monofilament. But yet, in other aspects, the first yarn can be a multifilament. In aspects where the first yarn is multifilament, such an exemplary yarn can have any desired filament count that is suitable for the specific application. For example and without limitations, the first yarn can have a filament count from greater than about 1 to about 50, including exemplary values of about 2, about 3, about 5, about 7, about 10, about 12, about 15, about 17, about 20, about 12, about 25, about 27, about 30, about 32, about 35, about 37, about 40, about 42, about 45, and about 47. It is also understood, however, that if it is desired for the specific application, the filament count can be greater than about 50, greater than about 80, or even greater than about 100.

In still further aspects, the one or more filaments of the first yarn have a size ranging from about 5 denier to about 1000 denier, including exemplary values of about 10 denier, about 20 denier, about 50 denier, about 100 denier, about 200 denier, about 300 denier, about 400 denier, about 500 denier, about 600 denier, about 700 denier, about 800 denier, and about 900 denier. Again, it is understood that the specific filament size can be determined based on the desired application.

In still further aspects, the one or more filaments of the first yarn can be produced by any method known in the art and suitable for the desired application. For example, and without limitations, the one or more filaments of the first yarn can be melt-spun.

In still further aspects, if more than one additional yarns are present in the disclosed herein textile, such additional yarns can be the same or different. It is understood that such additional yarns can have the same or different composition or be the same or different type.

In still further aspects, the one or more additional yarns comprise a polyolefin, a polyester, a polyurethane, a thermoplastic polyurethane, a polyamide, or a combination thereof. It is understood that in such aspects, the polyester can comprise polyethylene terephthalate (PET) homopolymer and copolymers, polypropylene terephthalate (PPT) homopolymer and copolymers and polybutylene terephthalate (PBT) homopolymer and copolymers, and the like, including those that contain comonomers such as cyclohexanedimethanol, cyclohexanedicarboxylic acid, isophthalic acid, and the like. In yet other aspects, the polyolefin can comprise ultra-high molecular weight polyethylene, low-molecular-weight polyethylene, polypropylene, polytetrafluoroethylene, expanded polytetrafluorethylene, and polyvinylidene fluoride. In still further aspects, the polyamide can comprise nylon 6, nylon 66, nylon 666, nylon 610, nylon 512, nylon 11, or nylon 12, or a combination thereof. In yet still further aspects, the one or more additional yarns can also comprise polyethers, polyureas, copolymers thereof, or a combination thereof.

In certain aspects, the one or more additional yarns can comprise a fully-drawn yarn, spin-drawn yarn, low- or not-twisted yarn, twisted yarn, a flat yarn, a textured yarn, a high (HS) stretch textured yarn, a high-bulk textured yarn, a high shrinkage yarn, or any combination thereof.

In certain aspects, the one or more additional yarns can also comprise a composite fiber. It is understood that, as used herein, the composite fiber relates to a fiber that can comprise one or more different materials. In certain aspects, the composite fiber is a bicomponent fiber that can have any configuration, as for example, and without limitation, a side-by-side configuration, a core-sheath configuration, a segmented configuration, an islands-in-the-sea configuration, or any combination thereof.

It is understood that the one or more additional yarns have lower stretchability and elasticity than the first yarn. In still further aspects, the one or more additional yarns can be substantially rigid. In yet still further aspects, the one or more additional yarns can be substantially semi-rigid.

In still further aspects, the textile can also comprise a second yarn. In such aspects, the second yarn can comprise one or more filaments comprising a second polymer. In still further aspects, the first polymer in the first yarn has a higher elasticity than the second polymer in the second yarn. In still further aspects, the second polymer can comprise a polyester, a nylon, a polyolefin, or a combination thereof. For example, and without limitations, the second polymer can comprise polyethylene terephthalate (PET) homopolymer and copolymers, polypropylene terephthalate (PPT) homopolymer, and copolymers and polybutylene terephthalate (PBT) homopolymer and copolymers, and the like. In yet other aspects, the second polymer can comprise ultra-high molecular weight polyethylene, low-molecular-weight polyethylene, polypropylene, polytetrafluoroethylene, expanded polytetrafluorethylene, polyvinylidene fluoride. In still further aspects, the second polymer can comprise nylon 6, nylon 66, nylon 666, nylon 610, nylon 512, nylon 11, or nylon 12, or a combination thereof. In yet still further aspects, the second yarn can be the same or different from the one or more additional yarns.

In still further aspects, the second yarn can cover the first yarn to form a multi-component yarn. In such exemplary and unlimiting aspects, the first yarn can form a core, and the second yarn can form a sheath. In some aspects, the second yarn can be monofilament., While in other aspects, the second yarn can be multifilament. In aspects where the second yarn is multifilament, such a yarn can have a filament count from greater than 1 to about 150, including exemplary values of about 2, about 5, about 7, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, and about 145. It is also understood, however, that if it is desired for the specific application, the filament count can be greater than about 150, greater than about 200, or even greater than about 300 if desired.

In still further aspects, the one or more filaments of the second yarn have a size ranging from about 5 denier to about 1000 denier, including exemplary values of about 10 denier, about 20 denier, about 50 denier, about 100 denier, about 200 denier, about 300 denier, about 400 denier, about 500 denier, about 600 denier, about 700 denier, about 800 denier, and about 900 denier. Again, it is understood that the specific filament size can be determined based on the desired application.

In still further aspects, the multi-component yarn can be single-covered or double-covered. In such exemplary and unlimiting aspects, if the multi-component yarn is double covered, two second yarns cover the first yarn in the opposite direction.

In still further aspects, the second yarn can be of any type. For example, and without limitations, the second yarn can comprise a fully-drawn yarn, spin-drawn yarn, low- or not-twisted yarn, twisted yarn, a flat yarn, a textured yarn, a high stretch (HS) textured yarn, a high-bulk textured yarn, a high shrinkage yarn, or any combination thereof.

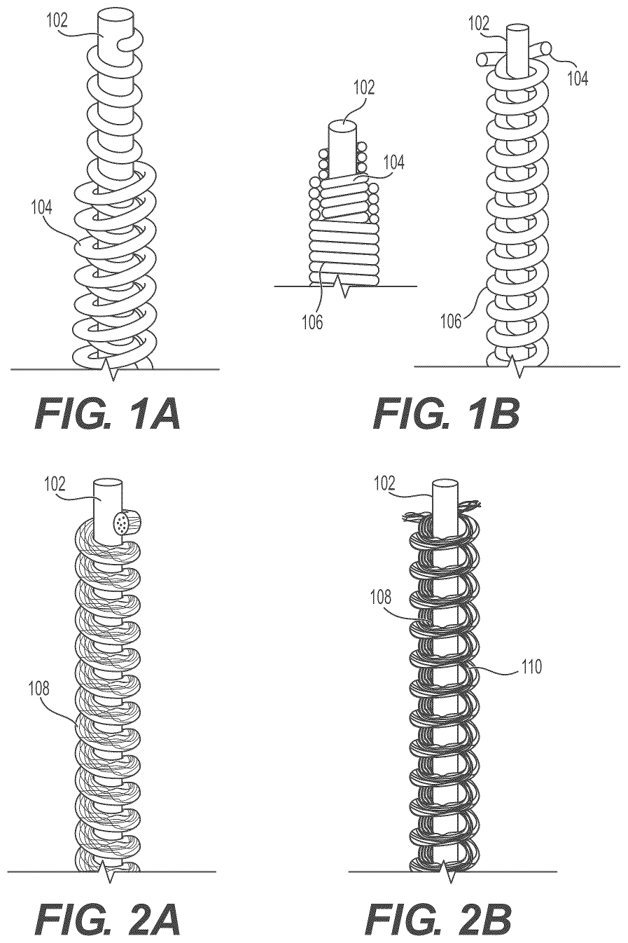

Some exemplary multi-component yarns are shown in FIGS. 1A-3B. For example, FIG. 1A shows a monofilament first yarn 102 covered with a monofilament second yarn 104. It also shows the beginning of the double covering by the second yarn. Additional more detailed schematics of the double covering are shown in FIG. 1B, wherein the monofilament first yarn 102 is covered with the monofilament second yarn 104 in one direction and then covered again with the monofilament second yarn 106 in a direction opposite from the direction of the second yarn 104 to form a double-covered multi-component yarn.

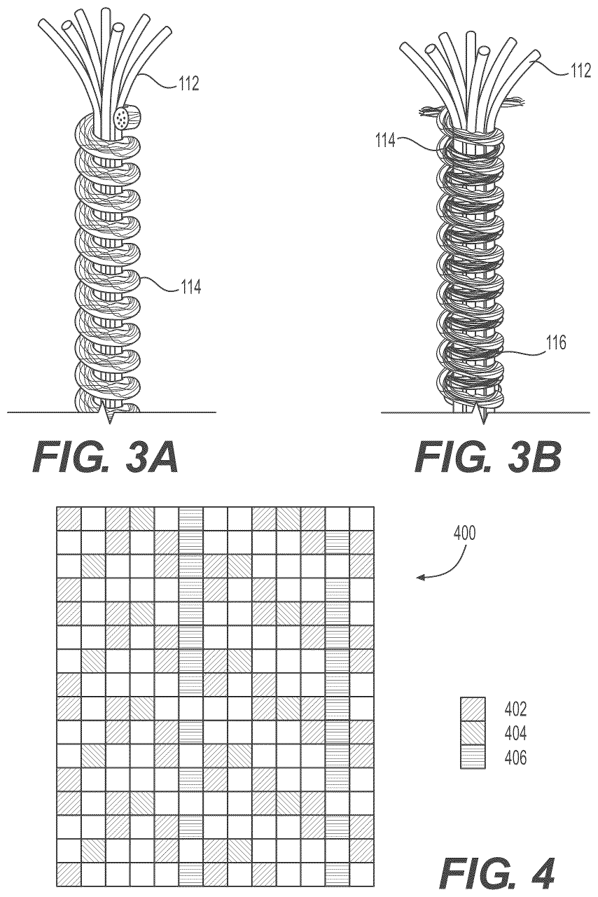

FIG. 2A similarly shows an exemplary schematic of a single covered multi-component yarn comprising the monofilament first yarn 102 and a multifilament second yarn 108. FIG. 2B shows a double covering of the monofilament first yarn 102 with the multifilament yarns 108 and 110 wrapped in the opposite directions.

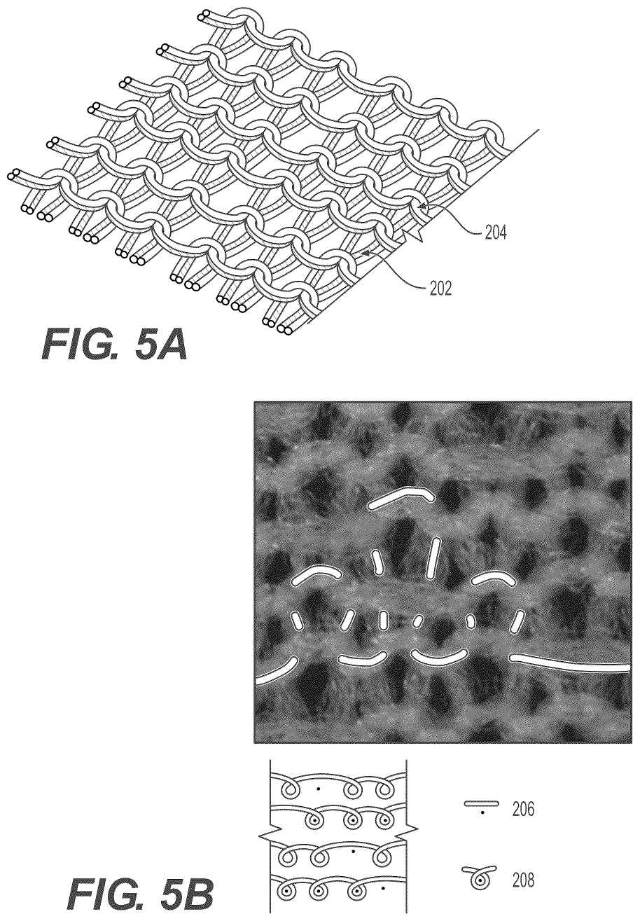

FIG. 3A shows a single covered multifilament first yarn 112 with a multifilament second yarn 114. FIG. 3B shows the double covering of the multifilament first yarn 112 with the multifilament second yarns 114 and 116 wrapped in the opposite directions.

In still further aspects, the textile disclosed herein can have any desired construction. In some aspects, the textile can have a knitted, woven, or braided construction.

In such aspects, if the textile is knitted, such a textile can comprise a simple single jersey knit construction; a single jersey knit with knit-miss pattern construction; a single jersey knit with knit-tuck pattern construction; tricot knit, locknit, satin knit, reveres locknit, sharkskin knit, purl knit, rib stitch knit, interlock stitch knit, double knit, warp knit, raschel knit cable knit, bird's eye knit, pontell knit, intarsia knit, jacquard knit, knitted terry textile, knitted velour textile, sliver knit, fleece knit, or any combination thereof. In yet other aspects, the textile can have a warp, weft, or crochet-knitted construction. Also, the weft yarn can comprise the first yarn that can be covered or non-covered.

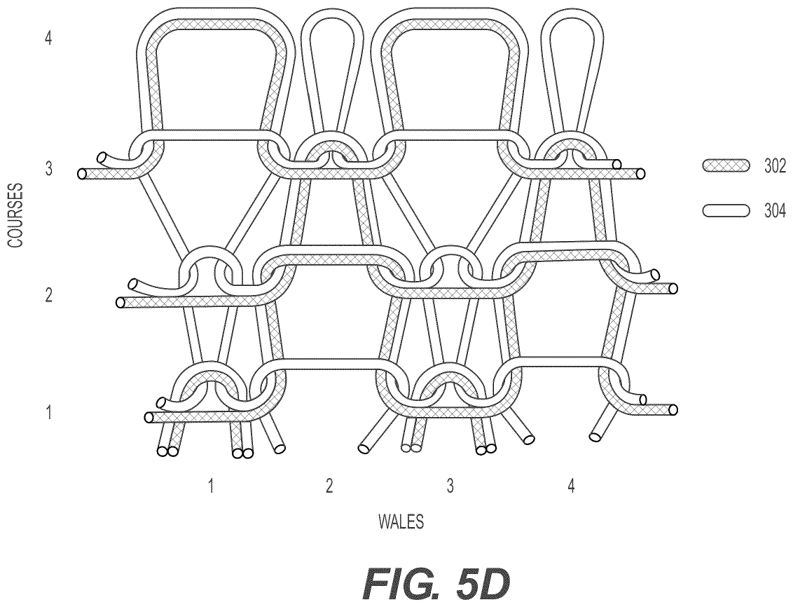

Some exemplary constructions are shown in FIGS. 5A-5D. For example, as shown in FIG. 5A, the textile can have a simple single jersey knit construction. In such exemplary aspects, the multi-component yarn 202 comprising the first yarn (for example, TPU) as a core and the second yarn (for example, PET) as a shell is knitted in a “plaiting arrangement” with the one or more additional yarns 204 (for example, a fine flat or twisted multifilament yarn). In these exemplary aspects, the flat or twisted yarn can be from about 10 denier to about 200 denier, including exemplary values of about 20 denier, about 50 denier, about 70 denier, about 100 denier, about 120 denier, about 150 denier, and about 170 denier, with a filament count in the yarn ranging from about 10 to about 100. FIG. 5B shows an exemplary single jersey knit with a knit-miss pattern, where 206 shows a miss stitch and 208 is a knit stitch. The multi-component yarns 212 and the one or more additional yarns 214 can be placed side by side. In certain aspects, such a pattern can have variations ranging from knit-knit-miss to knit-knit-knit-miss stitch combination, as shown in FIG. 5C, wherein 210 is a tuck stitch, and 208 is a knit stitch, and the multi-component yarn 212 and the one or more additional yarns 214 are placed side by side.

FIG. 5D shows a knit and tuck combination that involves feeding two yarns in a plating relationship to needles having forward hooks. An exemplary covered TPU core yarn 302 is fed at a high level and is received only by needles selected to that height. One or more additional yarns 304, for example, a fine yarn having a size of 10 denier to 100 denier, including exemplary values of about 20 denier, about 30 denier, about 40 denier, about 50 denier, about 60 denier, about 70 denier, about 80 denier, and about 90 denier, is fed at a lower level and is received and knitted by every needle. At the first two feeders, odd needles (1 &3) knit only the thin yarn, and even needles (2 & 4) knit plated loops. At the next two feeders, the sequence can be reversed.

In still further aspects, when the textile has a knitted construction (for example, weft, warp, or crochet-knit) and when the second yarn is not present, the first more elastomeric yarn is used in a weft direction of the textile. In such exemplary aspects, the warp direction can comprise the one or more additional yarns. In yet still further exemplary aspects, when the weft direction of the textile comprises the first yarn (without the presence of the second yarn, e.g., not the multi-component yarn), the warp direction can comprise a combination of the one or more additional yarns and the first yarn placed side by side or in any other desired pattern.

In still further aspects, when the second yarn is present, e.g., wherein the multi-component yarn is present, it is positioned in a warp direction. In such exemplary aspects, the warp direction can also comprise the one or more additional yarns. In such aspects, the multi-component yarn and the one or more additional yarns are present in a ratio from about 1:2 to about 1:10, including exemplary values of about 1:3, 1:4, 1:5, 1:6, 1:7, 1:8, and 1:9.

In some exemplary and unlimiting aspects, when the textile has a crochet knitting construction, a warp direction of such a textile can comprise the first yarn. Yet, in such aspects, the warp direction can further comprise the one or more additional yarns in a ratio from about 1:2 to about 1:10, including exemplary values of about 1:3, 1:4, 1:5, 1:6, 1:7, 1:8, and 1:9. In yet further aspects, the weft yarn can comprise the first yarn that can be covered or non-covered.

While in still further aspects, when the first yarn is not covered with the second yarn and the construction is a weft-knit, the textile can comprise plaiting of the first yarn and the one or more additional yarns.

In still further aspects, the textile can have a woven construction. The woven base layer is understood to be distinguishable from knitted fabrics in that the woven base layer is constructed by interlacing or weaving lengthwise and crosswise at least two warp and weft yarns to create the woven base layer. In certain aspects, the woven base layer does not have a great degree of stretchability due to the nature of its construction of interlaced warp and weft yarn unless the construction comprises stretchable yarns. This is in contrast to a knitted textile, which is made from interlocking loops that impart a greater degree of stretchability to the resulting knitted textile, regardless of whether or not the yarns themselves are stretchable. The stretchability of the knitted textile can be further improved by the use of elastomeric yarns, as disclosed herein.

In still further aspects disclosed herein, the woven construction can comprise a plain weave, derivatives of plain weave, a rib weave, a basket weave, a twill weave, a satin weave, a dobby weave, a jacquard weave, an extra yarn weave, a pique weave, a double cloth textile, a crepe weave, a leno weave, a pile weave, a triaxial weave, or a combination thereof.

In certain aspects, when the multi-component yarn is not present, e.g., where only the first yarn is present, a weft direction of the textile comprises the first yarn. In such exemplary and unlimiting aspects, the warp direction can comprise the one or more additional yarns. Yet, in still further exemplary aspects, when the first yarn is present in the weft direction, the warp direction can comprise a combination of the one or more additional yarns and the first yarn.

In still further aspects, when the textile is woven, and the first yarn is covered with the second yarn, i.e., forming the multi-component yarn, a warp direction of the textile comprises the multi-component yarn and the one or more additional yarns in a ratio from about 1:2 to about 1:10, including exemplary values of about 1:3, 1:4, 1:5, 1:6, 1:7, 1:8, and 1:9.

In some exemplary aspects, when the multi-component yarn is present in the woven textile, such a textile can further comprise a third yarn that is different from the first yarn. In still further aspects, the third yarn can also be different from the one or two additional yarns. In still further aspects, the third yarn can comprise a third polymer comprising a polyester, a nylon, a polyolefin, polyurethane, or a combination thereof. In still further aspects, any of the disclosed above polyester, nylons, polyolefins, polyurethanes, or a combination thereof can be used to form the third yarn.

In yet still further aspects, the third yarn can also comprise a fully-drawn yarn, spin-drawn yarn, low- or not-twisted yarn, twisted yarn, a flat yarn, a textured yarn, a high-bulk textured yarn, a high shrinkage yarn, or any combination thereof. In yet still further aspects, the third yarn can is the textured yarn.

In still further aspects, when the multi-component yarn is present in the woven textile, a weft direction of such a textile can comprise the one or more additional yarns.

In some aspects, the woven textile can be formed such that the multi-component yarn is provided from a first beam, the one or more additional yarns are provided from a second beam, and the third yarn, if present, is provided from a third beam. In such exemplary aspects, the multi-component yarn woven in the warp direction is present under a tension that is different from the tension of the one or more additional yarns and/or tension of the third yarn. In some aspects, the tension of the multi-component yarn is lower than the tension of the one or more additional yarns and/or the tension of the third yarn. Yet, in other aspects, the tension of the multi-component yarn in the warp direction can be greater than the tension of the one or more additional yarns and/or tension of the third yarn in the warp direction. In such exemplary aspects, the multi-component yarn is woven, for example, under higher tension, it can spring back for recovery to an original dimension after weaving is complete to allow additional stretchability to the textile. Yet, in other exemplary aspects, when the multi-component yarn is woven in the warp direction, for example, under lower tension than other yarns, it can also provide an additional stretch of the textile when the weaving is complete. In still further aspects, the third yarn can be woven separately in the warp direction from the multi-component yarn and/or the one or more additional yarns.

In still further exemplary aspects, the third yarn can form a longer float or raised feature/s on a surface of the textile when compared to a substantially identical reference textile in the absence of the third yarn. In such exemplary aspects, the woven textile can have a higher surface area compared to a substantially identical reference textile in the absence of the third yarn. These features can be very useful in some medical devices where a high surface of the textile is needed (for example, in grafts, in implantable devices, and the like).

In still further aspects, the textile can have a predetermined weave pattern comprising an alternating arrangement of the multi-component yarn side-by-side with the one or more additional yarns in a warp direction. In such aspects, the one or more additional yarns in the warp direction are the textured yarn, a high (HS) stretch textured yarn, the high-bulk textured yarn and/or a high shrinkage yarn. Yet, in other aspects, the one or more additional yarns in the weft direction can also be a flat or twisted yarn, textured yarn, a high (HS) stretch textured yarn, the high-bulk textured yarn and/or a high shrinkage yarn.

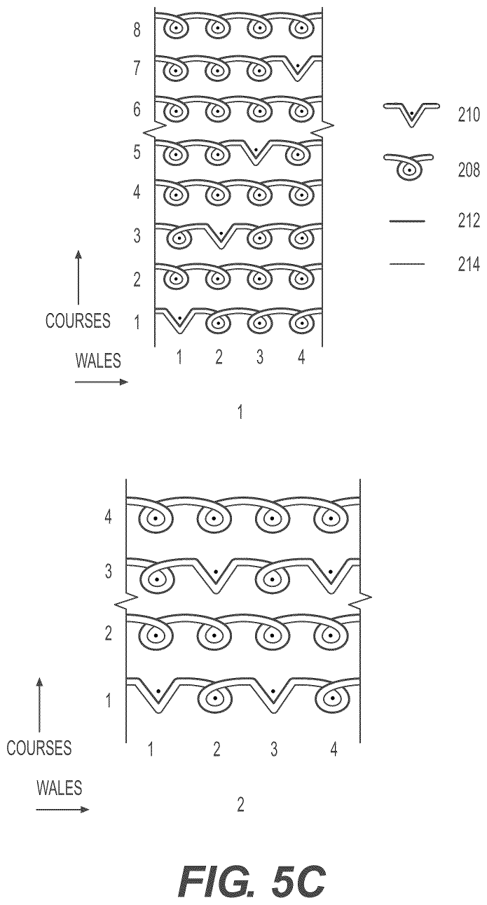

For example, FIG. 4 shows an exemplary and unlimiting design of textile 400, wherein the exemplary multi-component yarn with a TPU core 402 is woven with a textured yarn 404 and a high-bulk textured yarn 406 in the warp direction.

In still further exemplary and unlimiting aspects, in the woven textiles, a warp density of the multi-component yarn can be from about 10 ends/inch to about 200 ends/inch, including exemplary values of about 20 ends/inch, about 50 ends/inch, about 70 ends/inch, about 100 ends/inch, about 120 ends/inch, about 150 ends/inch, and about 170 ends/inch.

In still further aspects, a warp density of the one or more additional yarns comprising a textured yarn, a high (HS) stretch textured yarn, the high-bulk textured yarn and/or a high shrinkage yarn is about 10 ends/inch to about 200 ends per inch, including exemplary values of about 20 ends/inch, about 50 ends/inch, about 70 ends/inch, about 100 ends/inch, about 120 ends/inch, about 150 ends/inch, and about 170 ends/inch.

In yet still further aspects, a weft density is from about 10 picks/inch to about 200 picks/inch, including exemplary values of about 20 picks/inch, about 50 picks/inch, about 70 picks/inch, about 100 picks/inch, about 120 picks/inch, about 150 picks/inch, and about 170 picks/inch.

The permeability of the woven textile can be adjusted by varying the pore sizes of the woven textile as defined by adjacent warp and weft yarns. This can be accomplished by the selection of the yarns and/or by varying the ends per inch and the picks per inch of the woven textile. The permeability, as needed, can also be further adjusted by using any of the disclosed herein weave patterns. In still further aspects, the pore size in braids can be controlled by the selection of yarns, i.e., yarn size, number of ends, pick density, and braid pattern. In knitted fabrics, the pore size can be further controlled by the gauge used in the knitting machine that specifies the number of yarns per inch. i.e. 40 gauge=40 yarns per inch. Along with the fabric forming methods such as weaving, knitting, and/or braiding, certain post-processing, such as heat treatment at elevated temperatures, can be used to adjust the permeability or pore size.

In still further aspects, the textile can have a braided construction. In such exemplary aspects, the braided constructions can be biaxial, triaxial, unidirectional, or a combination thereof. In still further aspects, the braided construction can comprise a core and a sheath.

It is understood that the braided construction can comprise three or more yarns intertwined in such a way that no two yarns are twisted around one another. In still further aspects, the braided construction can also be referred to as a family of textiles continuously woven on the bias. It is further understood that the braided construction of the textile can be manufactured and used as a freestanding textile with a constant braid angle (the acute angle measured from the axis of the braid to the axis of the bias yarns) for a given diameter.

It is further understood that while the braided construction, similarly to the woven construction, can have yarns mechanically interlocked with one another because the yearns are also continuous, the braid can have a natural mechanism that evenly distributes load throughout the structure and thus makes the braided construction impact resistant that can be useful in various applications.

In such exemplary aspects, if the second yarn is not present, the core of the braided construction comprises the first yarn, and the sheath comprises the one or more additional yarns. Yet, in other aspects, when the second yarn is present, the sheath comprises the multi-component yarn. In such exemplary aspects, the braided construction can further comprise an additional first yarn that is not covered by the second yarn, and this additional first yarn can be present in the core of the braided construction.

In still further aspects, the textile can have any desired thickness. For example, and without limitations, In still further aspects, the textile can have a thickness from about 0.1 mm to about 10 mm, including exemplary values of about 0.2 mm, about 0.3 mm, about 0.4 mm, 0.5 mm, about 0.6 mm, about 0.7 mm, about 0.8 mm, about 0.9 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 8.5 mm, about 9 mm, and about 9.5 mm. In yet other aspects, the first textile thickness can have any values between any two foregoing values.

In still further aspects, the textile can have any desired length. For example and without limitations, the textile can have a length from about 1 mm to about 100 mm, including exemplary values of about 2 mm, about 5 mm, about 7 mm, about 10 mm, about 12 mm, about 15 mm, about 17 mm, about 20 mm, about 22 mm, about 25 mm, about 27 mm, about 30 mm, about 32, mm, about 35 mm, about 37 mm, about 40 mm, about 42 mm, about 45 mm, about 47 mm, about 50 mm, about 52, mm, about 55 mm, about 57 mm, about 60 mm, about 62 mm, about 65 mm, about 67 mm, about 70 mm, about 72, mm, about 75 mm, about 77 mm, about 80 mm, about 82 mm, about 85 mm, about 87 mm, about 90 mm, about 92, mm, about 95 mm, and about 97 mm. It is understood that these values are exemplary and not limiting and that textiles can have any length values between any two foregoing values. It is further understood that the length of the textile can be about 150 mm, about 200 mm, or about 1 m if the specified application requires it.

In still further aspects, the textile can have any desired width. In still further aspects, wherein the textile can have a width from about 1 mm to about 100 mm, including exemplary values of about 2 mm, about 5 mm, about 7 mm, about 10 mm, about 12, mm, about 15 mm, about 17 mm, about 20 mm, about 22 mm, about 25 mm, about 27 mm, about 30 mm, about 32, mm, about 35 mm, about 37 mm, about 40 mm, about 42 mm, about 45 mm, about 47 mm, about 50 mm, about 52, mm, about 55 mm, about 57 mm, about 60 mm, about 62 mm, about 65 mm, about 67 mm, about 70 mm, about 72, mm, about 75 mm, about 77 mm, about 80 mm, about 82 mm, about 85 mm, about 87 mm, about 90 mm, about 92, mm, about 95 mm, and about 97 mm. It is understood that these values are exemplary and not limiting and that textiles can have any width values between any two foregoing values. It is further understood that the first width can be about 150 mm, about 200 mm, or even about 1 m if the specified application requires it.

In still further aspects, the textile disclosed herein can exhibit elastic recovery of at least about 80% from an original dimension, at least about 85% from an original dimension, at least about 90% from an original dimension, at least about 95% from an original dimension, at least about 99% from an original dimension, or even 100% recovery from an original dimension.

In still further aspects, the disclosed textile can be stretched while exhibiting disclosed above elastic recovery along the longitudinal, the transverse axis, or across of the textiles. In still further aspects, when the textile has braided or knitted constructions, the elastic recovery can be in the axial direction or in bias directions as well. In such aspects, the textile can have an ultimate elongation along a length or width of about 50% to about 700%, including exemplary values of about 100%, about 150%, about 200%, about 250%, about 300%, about 350%, about 400%, about 450%, about 500%, about 550%, about 600%, and about 650%. In yet other aspects, the first polymer can have an ultimate elongation value between any two foregoing values. It is further understood that the textiles disclosed herein can have the same or different ultimate elongation measured along the textile width or length.

In still further aspects, when any of the disclosed herein textiles are compressed across its thickness, they can exhibit compressibility of about 10 to about 50%, including exemplary values of about 15%, about 20%, about 25%, about 30%, about 35%, about 40%, and about 45% as measured according to standard methods and at standard pressures.

In still further aspects, the fibers present in any of the disclosed herein yarns can have any diameter suitable for the desired application. In certain aspects, the fiber can have a diameter from about 1 μm to about 25 μm, including exemplary values of about 2 μm, about 5 μm, about 7 μm, about 10 μm, about 12 μm, about 15 μm, about 17 μm, about 20 μm, and about 22 μm. It is understood that the fiber can have any diameter between any two foregoing values. For example, and without limitation, the fiber can have a diameter from about 3 μm to about 8 μm, or from about 11 μm to about 22 μm, or from about 15 μm to about 25 μm

It is further understood that the aspects described herein comprise yarns that are biocompatible. Also disclosed herein are aspects that comprise yarns comprising permanent implant grade polymers. In still further aspects, the permanent implant grade polymers can include but are not limited to polyesters, co-polyesters, ultra-high molecular weight polyethylene, polyethylene, polypropylene, polytetrafluoroethylene, expanded polytetrafluorethylene, polyvinylidene fluoride, polyurethane, polyethers, polyureas, nylon, copolymers thereof, or a combination thereof.

In still further aspects, the textiles disclosed herein can exhibit high stretch, self-conforming to a substrate, predetermined suture retention, a predetermined cloth density retention, a pore size retention, thickness retention, or a combination thereof.

In still further aspects, the textiles can further comprise an active ingredient, wherein the active ingredient can comprise a pharmaceutical agent, a therapeutic agent, an antimicrobial agent, or any combination thereof.

In still further aspects, the textiles disclosed herein can be dip-coated, spray-coated, kiss-coated or laminated with films or membranes any materials that are suitable for the desired application. For example, the textile can be coated with a polymer, a drug, a hydrophobicity/hydrophilicity agent, a lubricious polymer, or any combination thereof.

In still further aspects, the polymer used for coating can comprise a thermoplastic polyurethane, silicone, or any combination thereof. It is understood that in some exemplary and unlimiting aspects, the coating can be used to form an impermeable barrier. Again, in such exemplary and unlimiting aspects, such coating can at least partially cover the pores on the textile structure while still maintaining the stretchability/elasticity of the construction

In still further aspects, the textile disclosed herein can be at least partially biostable. It is understood that the term “biostable” refers to the textile that remains at least partially intact over the period that the medical article and/or device is intended to remain within the body. Yet, in other aspects, the textile can be at least partially biodegradable. The term “biodegradable” as used herein can refer to the textile that at least partially degrades over the period which the medical article and/or device is intended to remain within the body, for example, due to dissolution or chemical breakdown, etc.

It is understood that the variations in the polymer content can affect the physical and chemical properties of the textile, such as mechanical strength, hardness, Surface tack, elasticity, compressibility, permeability, water diffusivity, therapeutic agent diffusivity (where present), degradation rate (where biodegradable), and hydrophobic/hydrophilic nature (influencing, for example, wettability, as well as water diffusivity and therapeutic agent diffusivity, if present), and the like.

In still further aspects, the textile is configured to be sutured. It is understood that any known in the art sutures can be used. In certain exemplary and unlimiting aspects, the suture is a textile filament. In yet other aspects, the suture is any filament that can be used for the desired application.

Medical Article and/or Device

Also disclosed herein are articles comprising any of the disclosed herein textiles. Such articles can comprise any medical device that has a textile component and is configured to be in contact with a subject's body and/or body liquids. In yet still further aspects, such articles can comprise implantable devices. In yet other aspects, the articles can comprise a tissue scaffold.

As can be appreciated, a wide variety of medical devices can be formed using the disclosed herein textiles. For example, devices such as closed-volume (hollow) medical devices, such as tubular articles (e.g., vascular and non-vascular grafts and stent grafts, including large and small vascular grafts such as coronary artery bypass grafts, peripheral vascular grafts and endovascular grafts, other tubular structures such as biliary, urethral, ureteral, intestinal and esophageal tubular structures, etc.), as well as various open-volume medical devices such as vascular and non-vascular patches (e.g., patches for wound healing, patches for hernia repair and patches for the gastrointestinal tract and the urogenital system) can be formed. Further examples of medical devices include vascular and non-vascular tissue scaffolding, vascular and non-vascular closure devices, for example, devices for closure of the peripheral and arterio-venous fistula, sutures, meshes, valve leaflets for heart valves and venous valves, vascular access devices including vascular access ports and arteriovenous access grafts (e.g., devices which are utilized to give frequent arterial and/or venous access such as for antibiotics, total parental nutrition, intravenous fluids, blood transfusion, blood sampling, or arterio-venous access for hemodialysis, and so forth), embolic filters (e.g., distal protection filters), uterine slings, fabric to join LVADs (left ventricular assist devices) and TAHS (total artificial hearts) to human arteries, and so forth.

In some aspects, the medical articles and/or devices comprising the disclosed herein textiles can be suitable for long-term implantation. As used herein, the term “long-term” implantation means implantation periods greater than 30 days, for example, ranging from 1 month to 3 months to 6 months to 12 months to 24 months or even longer, including the remaining lifetime of the patient. In instances where hollow (including tubular) medical devices are provided to reinforce, repair, or replace a body lumen (e.g., stents, grafts, stent-grafts, patches, etc.), their dimensions may be tailored to approximate the dimensions of all or a portion of the body lumen. Examples of body lumens include lumens of the cardiovascular system such as the heart, arteries, and veins (e.g., coronary, femoral, aorta, ilial, carotid, and vertebrobasilar arteries), lumens of the genitourinary system such as the urethra (including prostatic urethra), bladder, ureters, vagina, uterus, spermatic and fallopian tubes, the nasolacrimal duct, the eustachian tube, lumens of the respiratory tract, such as the trachea, bronchi, nasal passages and sinuses, lumens of the gastrointestinal tract such as the esophagus, gut, duodenum, small intestine, large intestine, colon, biliary and pancreatic duct systems, lumens of the lymphatic system, and so forth.

Hence, hollow medical devices (including any tubular shape. Such as those having circular and elliptical cross-sections) for use in the present invention may vary widely in diameter, for example, ranging from 0.5 mm to 1 mm to 2 mm to 5 mm to 10 mm to 20 mm to 50 mm or more in diameter. For instance, tubular articles having diameters ranging from 0.5 to 2 mm may be employed for microvascular work and conduits for nerve regeneration, those having diameters ranging from 2 to 4 mm may be employed for coronary bypass, those having diameters ranging from 2 to 10 mm, may be employed peripheral vascular grafts, those having diameters ranging from 20 to 50 mm and above may be employed for endovascular and endoluminal vascular grafts, other tubular prosthesis such as esophageal and colonic prosthesis, and so forth.

In yet other aspects, the textiles disclosed herein can be used to attend to issues associated with a paravalvular leak (PVL). The PVL is a complication associated with the implantation of a prosthetic heart valve. PVL refers to blood flowing through a channel between the structure of the implanted valve and cardiac tissue as a result of a lack of appropriate sealing. The majority of PVL are crescent, oval, or roundish-shaped, and their track can be parallel, perpendicular, or serpiginous. Transcatheter Heart Valve (THV) procedures generally use either a substantially inelastic woven cloth or a stretchable knitted cloth for PVL sealing. It is understood that the stretchability of the textile can be an important factor as it helps in reducing stress on a tissue to which the medical device comprising the textile is attached.

With the next generation of THV frame designs that have changing frame dimensions, one of the requirements is to have the PVL seal cloth and/or the frame inner cloth to adapt to the changing frame dimensions. Thus, there is a need for a cloth having controlled stretchability and a lower profile to provide improved compliance by reducing potential stresses at locations where the cloth is secured to a bodily lumen.

In some exemplary aspects, the textiles described herein can be used in an implantable medical device comprising: an annular frame having an inflow end, an outflow end, and a longitudinal axis; and a sealing element secured to the frame, wherein the sealing element comprises any of the disclosed herein textiles.

In certain exemplary and unlimiting aspects, disclosed herein is an implantable prosthetic valve comprising: an annular frame having an inflow end, an outflow end, and a longitudinal axis, wherein the annular frame is configured to expand and collapse; and a sealing element secured to the frame, wherein the sealing element comprises a textile comprising: a) a first yarn comprising one or more filaments comprising a first polymer; b) one or more additional yarns comprising a composition and/or properties different from the first yarn. In some aspects, the first yarn exhibits an elasticity higher than an elasticity of the one or more additional yarns. In certain aspects, the textile exhibits an ultimate elongation of about 50% to about 700% and is shape-adapting and biocompatible. In some implementations, the textile is configured to be inserted and/or implanted in a subject's body.

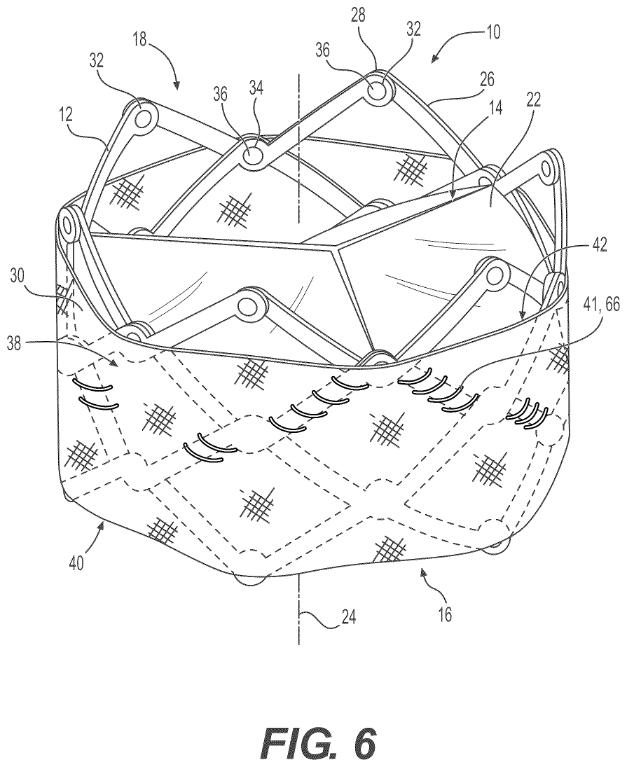

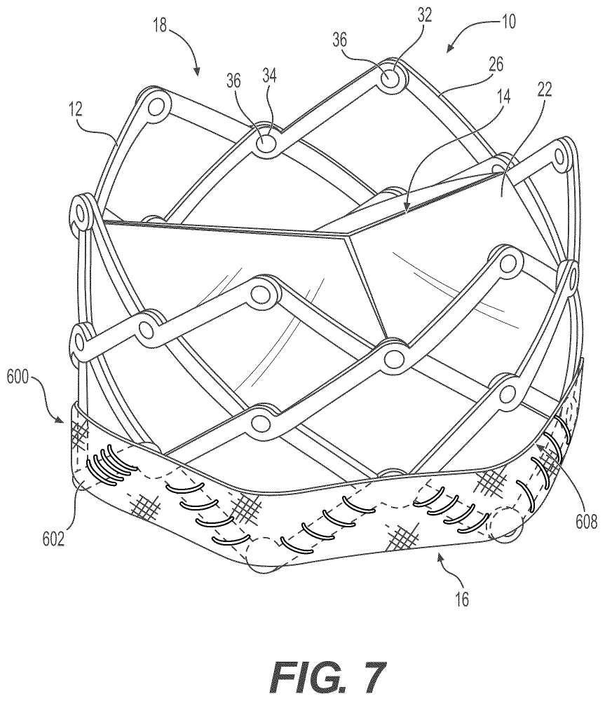

The exemplary aspects of the implantable medical device are shown in FIGS. 6-7. One exemplary and unlimiting aspect encompasses medical devices that are prosthetic heart valves. For example, and without limitation, FIG. 6 illustrates an exemplary aspect of a radially collapsible and expandable prosthetic valve 10 shown in its deployed, expanded configuration. The prosthetic valve can include an annular stent or frame 12, and a leaflet structure 14 situated within and coupled to the frame 12. The frame 12 can have an inflow end portion 16 and an outflow end portion 18. The leaflet structure can comprise a plurality of leaflets 22. In certain aspects, the leaflet structure can comprise three leaflets. In such exemplary aspects, such three leaflets can be arranged to collapse in a tricuspid arrangement similar to the aortic valve. Alternatively, the prosthetic valve can include two leaflets 22 configured to collapse in a bicuspid arrangement similar to the mitral valve, or more than three leaflets, depending upon the particular application. The prosthetic valve 10 can define a longitudinal axis 24 extending through the inflow end portion 16 and the outflow end portion 18.

The frame 12 can be made of various biocompatible materials, such as stainless steel or a nickel-titanium alloy (“NiTi”), for example, Nitinol. With reference to FIG. 6, the frame 12 can include a plurality of interconnected lattice struts 26 arranged in a lattice-type pattern and forming a plurality of apices 28 at the outflow end 18 of the prosthetic valve. The struts 26 can also form similar apices at the inflow end 16 of the prosthetic valve (which are covered by a skirt 30 described in greater detail below). The lattice struts 26 are shown positioned diagonally or offset at an angle relative to, and radially offset from, the longitudinal axis 24 of the prosthetic valve. It is understood that the configuration depicted in FIG. 6 is exemplary only, and in other aspects, the lattice struts 26 can be offset by a different amount than depicted in FIG. 6 or some or all of the lattice struts 26 can be positioned parallel to the longitudinal axis of the prosthetic valve.

The lattice struts 26 can be pivotably coupled to one another. In the illustrated aspect, for example, the end portions of the struts 26 forming the apices 28 at the outflow end 18 and at the inflow end 16 of the frame can have a respective opening 32. The struts 26 also can be formed with apertures 34 located between the opposite ends of the struts. Respective hinges can be formed at the apices 28 and at the locations where struts 26 overlap each other between the ends of the frame via fasteners 36, which can comprise rivets or pins that extend through the apertures 32, 34. The hinges can allow the struts 26 to pivot relative to one another as the frame 12 is expanded or contracted (or collapsed), such as during assembly, preparation, or implantation of the prosthetic valve 10. For example, the frame 12 (and, thus, the prosthetic valve 10) can be manipulated into a radially compressed or contracted (collapsed) configuration, coupled to a delivery apparatus, and inserted into a patient for implantation. Once inside the body, the prosthetic valve 10 can be manipulated into an expanded state and then released from the delivery apparatus. Additional details regarding the frame 12, the delivery apparatus, and devices and techniques for radially expanding and collapsing the frame can be found in U.S. Publication No. 2018/0153689, which is incorporated herein by reference. Additional details about such an exemplary prosthetic valve can also be found in U.S. Publication No. 2019/0046314, which is incorporated herein by reference.

As further illustrated in FIG. 6, the prosthetic valve 10 can include a sealing element configured as a skirt 30. The skirt 30, as shown herein, can comprise any textile materials disclosed above. It is understood that in some aspects, the skirt 30 is an outer skirt. In such aspects, another skirt (not shown) known as an inner skirt can be attached to the frame underneath of the outer skirt 30. However, in some aspects, the textile materials used in the outer skirt 30 can also be used in the inner skirt (not shown here). Yet, in some aspects, the inner skirt does not comprise the textile materials used in the outer skirt. In still further aspects, only one skirt is used. In such aspects, such a skirt can comprise any of the textile materials described herein.

The skirt 30 can be configured to establish a seal with the native tissue at the treatment site to reduce or prevent paravalvular leakage. The skirt 30 can include a main body portion 38 disposed about an outer circumference of the frame 12. In certain aspects, the textile, or the skirt shown herein, for example, can be secured to the frame with a fastener. The fastener can include sutures, pins, rivets, ultrasonic welding, laser welding, adhesive bonding, or any combination thereof. In this example, the skirt 30 can be secured to the frame by, for example, a plurality of sutures 41 extending in a zig-zag pattern along selected strut members 26 between a first edge portion (e.g., an inflow edge portion) 40 and a second edge portion (e.g., an outflow edge portion) 42 of the skirt 30. For example, in certain aspects, the skirt 30 can be sutured to the frame 12 along a suture line 66 corresponding to a scalloped edge defined by the leaflets 22, which can allow the valve to radially expand and contract (collapse) without interference from or pinching of the skirt. Further details regarding transcatheter prosthetic heart valves, including the manner in which the leaflets 22 can be coupled to the frame 12, can be found, for example, in U.S. Pat. Nos. 6,730,118, 7,393,360, 7,510,575, 7,993,394, and 8,652,202, which are incorporated herein by reference in their entireties.

It is understood that the skirt 30 comprising the disclosed herein textile materials provides better dimensional stability that helps in procedures dealing with joining the valve components together using sutures and laser cutting of components. The disclosed textile allows the skirt 30 to adapt to the changing frame dimensions and provides a low profile for such a skirt.

FIG. 7 illustrates a prosthetic valve 10, including another aspect of a sealing member or skirt 600. In the illustrated and unlimiting aspect, the skirt 600 comprises a textile material as described herein and configured as a fabric strip 602 having an edge portion 608. In certain exemplary aspects, the skirt 600 can be secured to struts 26 to form layers of the skirts (not shown). It is further understood that any configurations of the skirt coupling with the frame can be used depending on the desired application.

The textile material described herein can be used in any sealing element aspect and in any combination with any of the prosthetic valves and/or frame aspects. It is understood that a prosthetic heart valve can also include any of the textile materials described herein, or portions thereof, in any combination.

The textiles of the prosthetic sealing members described herein can be configured to promote a biological response in order to form a seal between the prosthetic valve and the surrounding anatomy. In certain configurations, the sealing elements described herein can be configured to form a seal over a selected period of time. In some aspects, the sealing members of the paravalvular sealing structure can be treated with one or more agents that inhibit the biological response to the sealing structures. For example, in certain exemplary aspects, the textiles of the present disclosure can be treated with heparin. In certain aspects, the amount or concentration of the agent(s) can be selected such that the agents are depleted after a selected period of time (e.g., days, weeks, or months) after valve implantation. As the agent(s) are depleted, the biological response to the textile or yarns or fibers of the sealing structures can increase such that a paravalvular seal forms gradually over a selected period of time. This can be advantageous in patients suffering from left atrial remodeling (e.g., due to mitral regurgitation) by providing an opportunity for the remodeling to reverse as regurgitation past the prosthetic valve is gradually reduced.

### Methods

The present disclosure also provides for a method comprising forming a textile comprising: a) a first yarn comprising one or more filaments comprising a first polymer; b) one or more additional yarns that have a composition and/or properties different from the first yarn; and c) optionally a second yarn comprising one or more filaments comprising a second polymer, wherein if the second yarn is present, the second yarn covers the first yarn to form a multi-component yarn such that the first yarn forms a core and the second yarn forms a sheath, wherein the first yarn exhibits an elasticity higher than an elasticity of the one or more additional yarns and of the second yarn if present; and wherein the textile exhibits an ultimate elongation of about 50% to about 700% and is shape-adapting and biocompatible, is configured to be inserted and/or implanted in a subject's body.

Any of the disclosed herein first yarns, second yarns, and the one or more additional yarns can be used. In some aspects, and as disclosed above, the step of forming can further include the use of any of the disclosed above third yarns.

In some aspects, the step of forming can comprise a step of weaving, knitting, braiding, or any combination thereof. It is further understood that any of the disclosed above structures can be formed.

The present disclosure also provides for a method of making an implantable medical device comprising: providing a sealing element of any of the disclosed herein textiles and securing the sealing element to an annular frame of the implantable medical device.

In still further aspects, any of the implantable medical devices disclosed herein can be formed by such methods. In still further aspects, any of the disclosed herein textiles can be used to form the sealing element of the device. In still further aspects, any methods commonly used to secure the sealing element to an annular frame can be used. For example, and without limitation, the sealing element can be secured to the annular frame of the device with a suture. Any known in the art sutures can be used. In some exemplary and unlimiting aspects, the sutures can comprise textile-based materials. However, it is understood that any sutures that are applicable to the desired applications can be utilized.

In still further aspects, the methods disclosed herein can comprise a step of impregnating any of the disclosed herein textile materials with a pharmaceutically active agent, depending on the desired application. In still further aspects, the methods disclosed herein can comprise a step of coating any of the disclosed herein textile materials with any known in the art materials that can provide for any additional desired properties.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for.

Unless indicated otherwise, parts are parts by weight, temperature is degrees C. or is at ambient temperature, and pressure is at or near atmospheric or full vacuum.

### EXAMPLE 1

In this example, a textile is formed with a TPU-covered yarn. First, a TPU polymer was extruded to desired filament size using melt spinning, forming either monofilament or multifilament yarns.

Table 1 shows various properties of TPU materials used for extrusion. In some examples, the TPU medical grade with 93-100A was used for extrusion.

The TPU yarn was then covered with PET multifilament yarn. A textured yarn with high bulk was used for covering to protect the TPU filament/s from direct contact with metal surfaces while processing on textile equipment. The covered TPU yarn was then knitted (warp or weft knitting), woven, or braided into textile construction. The elastic textile structure was then coated with TPU spray or dip coating.

TABLE 1
Physical properties of TPU.

| | | |
|---|---|---|
| Durometer range available | 75 Shore A-75 Shore D | D2240* |
| Water Absorption | 1.00% | D570* |
| Melt Flow | 2-26 g/10 min (205° C./3.26 kg) | D1238* |

Mechanical Properties Ranges (Example Ranges Shown)

| | | | |
|---|---|---|---|
| Durometer | 80A | 75D | |
| Ultimate Tensile | 5500-8000 | 4500-6500 | D638* |
| Strength (psi) | | | |
| Tensile (psi) | | | |
| @50% elongation | 500-700 | 4200-5000 | D638* |
| @100% elongation | 800-1000 | 4500-5200 | D638* |
| @200% elongation | 1920-2100 | 4900-5800 | |
| @300% elongation | 5200-6000 | | D638* |
| Ultimate Elongation (%) | 300-500 | 200-350 | D638* |

*ASTM standard

TPUs used for coating have properties shown in Table 2, TPU grade higher solid % (20%+) used for coating, enabling the lower tackiness of the TPU.

TABLE 2
TPU properties

| | | | ASTM |
|---|---|---|---|
| Physical Properties | TPU A | TPU B | STANDARD |
| Viscosity (cps) | | | |
| @8% solids | 100-800 | 200-1000 | |
| @20% solids | 10000-50000 | 15000-50000 | |
| Ultimate Tensile | 3000-10000 | 6000-10000 | D638/D882 |
| Strength (psi) | | | |
| Tensile (psi) | | | |
| @50% elongation | 250-600 | 450-850 | D638/D882 |
| @100% elongation | 400-1000 | 650-1200 | D638/D882 |
| @200% elongation | 700-2000 | 1000-2300 | |
| @300% elongation | 950-3500 | 1600-4000 | D638/D882 |
| Ultimate Elongation (%) | 600-1200 | 500-1100 | D638/D882 |

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A textile comprising: a) a first yarn comprising one or more filaments comprising a first polymer; b) one or more additional yarns comprising a composition and/or properties different from the first yarn; wherein the first yarn exhibits an elasticity higher than an elasticity of the one or more additional yarns the textile exhibits an ultimate elongation of about 50% to about 700% and is shape-adapting and biocompatible, and wherein the textile is configured to be inserted and/or implanted in a subject's body.

Example 2: The textile of any examples herein, particularly example 1, wherein the first polymer comprises a thermoplastic polyurethane (TPU), a polyurethane (PU), a thermoplastic elastomer (TPE), an elastomeric polyolefin, polybutylene terephthalate (PBT), silicone-based elastomer, or a combination thereof.

Example 3: The textile of any examples herein, particularly example 1 or 2, wherein the first polymer has a durometer from about 75 A to about 75 D.

Example 4: The textile of any examples herein, particularly examples 1-3, wherein the first polymer has an ultimate tensile strength between about 4500 psi to about 8000 psi.

Example 5: The textile of any examples herein, particularly examples 1-4, wherein the first polymer has an ultimate elongation between about 50% to about 700%.

Example 6: The textile of any examples herein, particularly examples 1-5, wherein the first polymer is the TPU.

Example 7: The textile of any examples herein, particularly examples 1-6, wherein the first yarn is a monofilament.

Example 8: The textile of any examples herein, particularly examples 1-6, wherein the first yarn is a multifilament.

Example 9: The textile of any examples herein, particularly example 8, wherein the first yarn has a filament count from greater than about 1 to about 50.

Example 10: The textile of any examples herein, particularly examples 1-9, wherein the one or more filaments of the first yarn has a size ranging from about 5 denier to about 1000 denier.

Example 11: The textile of any examples herein, particularly examples 1-10, wherein more than one additional yarn is present, these additional yarns are the same or different.

Example 12: The textile of any examples herein, particularly examples 1-11, wherein the one or more additional yarns comprise a fully-drawn yarn, spin-drawn yarn, low- or not-twisted yarn, twisted yarn, a flat yarn, a textured yarn, a high (HS) stretch textured yarn, a high-bulk textured yarn, a high shrinkage yarn, or any combination thereof.

Example 13: The textile of any examples herein, particularly examples 1-12, wherein the one or more additional yarns comprise a polyolefin, a polyester, a polyurethane, a thermoplastic polyurethane, a polyamide, or a combination thereof.

Example 14: The textile of any examples herein, particularly examples 1-13, wherein the one or more additional yarns are substantially rigid or semi-rigid.

Example 15: The textile of any examples herein, particularly examples 1-14, further comprising a second yarn comprising one or more filaments comprising a second polymer.

Example 16: The textile of any examples herein, particularly example 15, wherein the second yarn is different from the first yarn.

Example 17: The textile of any examples herein, particularly example 15 or 16, wherein the second yarn covers the first yarn to form a multi-component yarn such that the first yarn forms a core and the second yarn forms a sheath.

Example 18: The textile of any examples herein, particularly examples 15-17, wherein the first polymer has higher elasticity than the second polymer.

Example 19: The textile of any examples herein, particularly examples 15-18, wherein the second polymer comprises a polyester, a nylon, a polyolefin, or a combination thereof.

Example 20: The textile of any examples herein, particularly example 19, wherein the second polymer comprises polyethylene terephthalate (PET) homopolymer and copolymers, polypropylene terephthalate (PPT) homopolymer and copolymers and polybutylene terephthalate (PBT) homopolymer and copolymers, or any combination thereof.

Example 21: The textile of any examples herein, particularly examples 15-20, wherein the second yarn is a monofilament.

Example 22: The textile of any examples herein, particularly examples 15-20, wherein the second yarn is a multifilament.

Example 23: The textile of any examples herein, particularly example 22, wherein the second yarn has a filament count from greater than 1 to about 150.

Example 24: The textile of any examples herein, particularly examples 15-23, wherein the one or more filaments of the second yarn has a size ranging from about 5 denier to about 1000 denier.

Example 25: The textile of any examples herein, particularly examples 17-24, wherein the multi-component yarn is single covered or double covered.

Example 26: The textile of any examples herein, particularly example 25, wherein the multi-component yarn is double covered, two second yarns cover the first yarn in the opposite direction.

Example 27: The textile of any examples herein, particularly examples 15-26, wherein the second yarn comprises a fully-drawn yarn, spin-drawn yarn, low- or not-twisted yarn, twisted yarn, a flat yarn, a textured yarn, a high stretch (HS) textured yarn, a high-bulk textured yarn, a high shrinkage yarn, or any combination thereof.

Example 28: The textile of any examples herein, particularly examples 1-27, wherein the one or more filaments of the first yarn is melt-spun.

Example 29: The textile of any examples herein, particularly examples 1-28, wherein the textile has a knitted, woven, or braided construction.

Example 30: The textile of any examples herein, particularly example 29, wherein the textile comprises a simple single jersey knit construction; a single jersey knit with knit-miss pattern construction; a single jersey knit with knit-tuck pattern construction; tricot knit, locknit, satin knit, reveres locknit, sharkskin knit, purl knit, rib stitch knit, interlock stitch knit, double knit, warp knit, raschel knit cable knit, bird's eye knit, pontell knit, intarsia knit, jacquard knit, knitted terry textile, knitted velour textile, sliver knit, fleece knit, or any combination thereof.

Example 31: The textile of any examples herein, particularly example 29 or 30, wherein the textile has a warp, weft (circular or flat), or crochet-knitted construction.

Example 32: The textile of any examples herein, particularly example 29, wherein the textile has the woven construction.

Example 33: The textile of any examples herein, particularly example 32, wherein the woven construction comprises a plain weave, derivatives of plain weave, a rib weave, a basket weave, a twill weave, a satin weave, a dobby weave, a jacquard weave, an extra yarn weave, a pique weave, a double cloth textile, a crepe weave, a leno weave, a pile weave, a triaxial weave, or a combination thereof.

Example 34: The textile of any examples herein, particularly example 29, wherein the textile has the braided construction.

Example 35: The textile of any examples herein, particularly example 34, wherein the braided constructions are biaxial, triaxial, unidirectional, or a combination thereof.

Example 36: The textile of any examples herein, particularly example 32 or 33, wherein the second yarn is not present, a weft direction of the textile comprises the first yarn.

Example 37: The textile of any examples herein, particularly example 36, wherein the second yarn is not present, a warp direction of the textile comprises the one or more additional yarns.

Example 38: The textile of any examples herein, particularly example 36, wherein the second yarn is not present, a warp direction of the textile comprises a combination of the one or more additional yarns and the first yarn.

Example 39: The textile of any examples herein, particularly example 32 or 33, wherein the second yarn is present, a warp direction of the textile comprises the multi-component yarn and the one or more additional yarns in a ratio from about 1:2 to about 1:10.

Example 40: The textile of any examples herein, particularly example 39, wherein the warp direction of the textile further comprises a third yarn that is different from the first yarn.

Example 41: The textile of any examples herein, particularly example 40, wherein the third yarn comprises a third polymer comprising a polyester, a nylon, a polyolefin, polyurethane, or a combination thereof.

Example 42: The textile of any examples herein, particularly example 40 or 41, wherein the third yarn comprises a fully-drawn yarn, spin-drawn yarn, low- or not-twisted yarn, twisted yarn, a flat yarn, a textured yarn, a high-bulk textured yarn, a high shrinkage yarn, or any combination thereof.

Example 43: The textile of any examples herein, particularly example 42, wherein the third yarn is the textured yarn.

Example 44: The textile of any examples herein, particularly examples 39-43, wherein the second yarn is present, a weft direction of the textile comprises the one or more additional yarns.

Example 45: The textile of any examples herein, particularly examples 39-44, wherein the multi-component yarn is woven in the warp direction under a tension different than a tension of the one or more additional yarns woven in the warp direction.

Example 46: The textile of any examples herein, particularly example 45, wherein the multi-component yarn is woven in the warp direction under a tension higher than a tension of the one or more additional yarns woven in the warp direction.

Example 47: The textile of any examples herein, particularly example 45, wherein the multi-component yarn is woven in the warp direction under a tension lower than a tension of the one or more additional yarns woven in the warp direction.

Example 48: The textile of any examples herein, particularly examples 39-47, wherein the third yarn is woven separately in the warp direction from the multi-component yarn and/or the one or more additional yarns.

Example 49: The textile of any examples herein, particularly example 48, wherein the third yarn forms a longer float or raised feature/s on a surface of the textile when compared to a substantially identical reference textile in the absence of the third yarn.

Example 50: The textile of any examples herein, particularly example 49, wherein the textile has a higher surface area compared to a substantially identical reference textile in the absence of the third yarn.

Example 51: The textile of any examples herein, particularly examples 39-50, wherein the textile has a predetermined weave pattern comprising an alternating arrangement of the multi-component yarn side-by-side with the one or more additional yarns in a warp direction.

Example 52: The textile of any examples herein, particularly examples 39-51, wherein the one or more additional yarns in the warp direction are the textured yarn, a high (HS) stretch textured yarn, the high-bulk textured yarn, and/or a high shrink yarn.

Example 53: The textile of any examples herein, particularly examples 44-52, wherein the one or more additional yarns in the weft direction are flat or twisted yarns, textured yarn, a high (HS) stretch textured yarn, and/or the high-bulk textured yarn.

Example 54: The textile of any examples herein, particularly examples 39-53, wherein a warp density of the multi-component yarn is from about 10 ends/inch to about 200 ends/inch.

Example 55: The textile of any examples herein, particularly examples 51-54, wherein a warp density of the one or more additional yarns comprising a textured yarn, a high (HS) stretch textured yarn, the high-bulk textured yarn and/or high shrinkage yarn is about 10 ends/inch to about 200 ends per inch.

Example 56: The textile of any examples herein, particularly examples 49-55, wherein a weft density is from about 10 picks/inch to about 200 picks/inch.

Example 57: The textile of any examples herein, particularly example 31, wherein the textile does not comprise the second yarn and has the warp-knitted construction, a warp direction of the textile comprises the first yarn and the one or more additional yarns in a ratio from about 1:2 to about 1:10.

Example 58: The textile of any examples herein, particularly example 31, wherein the textile has a crochet knitting construction, a warp direction of the textile comprises the first yarn.

Example 59: The textile of any examples herein, particularly example 58, wherein the warp direction further comprises the one or more additional yarns in a ratio from about 1:2 to about 1:10.

Example 60: The textile of any examples herein, particularly example 31, wherein the second yarn is present, and the textile has the warp-knitted construction, a warp direction of the textile comprises the multi-component yarn and the one or more additional yarns in a ratio from about 1:2 to about 1:10.

Example 61: The textile of any examples herein, particularly examples 57-60, wherein a weft direction of the textile comprises the one or more additional yarns.

Example 62: The textile of any examples herein, particularly example 31, wherein the textile does not comprise the second yarn and has the weft-knitted construction, the textile comprises plaiting of the first yarn and the one or more additional yarns.

Example 63: The textile of any examples herein, particularly example 34 or 35, wherein the braided construction comprises a core and a sheath.

Example 64: The textile of any examples herein, particularly example 63, wherein if the second yarn is not present, the core of the braided construction comprises the first yarn, and the sheath comprises the one or more additional yarns.

Example 65: The textile of any examples herein, particularly example 63, wherein the second yarn is present, the sheath comprises the multi-component yarn.

Example 66: The textile of any examples herein, particularly example 65, wherein the braided construction further comprises an additional first yarn that is not covered by the second yarn, wherein the core comprises the additional first yarn.

Example 67: The textile of any examples herein, particularly examples 1-66, wherein the textile exhibits elastic recovery of at least about 80%.

Example 68: The textile of any examples herein, particularly examples 1-67, wherein the textile exhibits high stretch, self-conforming to a substrate, a predetermined suture retention, a predetermined cloth density retention, a pore size retention, a thickness retention, or a combination thereof.

Example 69: An article comprising the textile of any examples herein, particularly examples 1-68.

Example 70: The article of any examples herein, particularly example 69, wherein the article comprises an implantable device.

Example 71: The article of any examples herein, particularly examples 69-70, wherein the article is in substantial contact with a subject's body and/or subject's body liquids.

Example 72: A medical device comprising the textile of any examples herein, particularly examples 1-66.

Example 73: The medical device of any examples herein, particularly example 72, wherein the device is implantable.

Example 74: The medical device of any examples herein, particularly examples 72-73, wherein the device is configured to be inserted in a subject's body.

Example 75: A method comprising forming a textile comprising: a) a first yarn comprising one or more filaments comprising a first polymer; b) one or more additional yarns that have a composition and/or properties different from the first yarn; and c) optionally a second yarn comprising one or more filaments comprising a second polymer, wherein if the second yarn is present, the second yarn covers the first yarn to form a multi-component yarn such that the first yarn forms a core and the second yarn forms a sheath, wherein the first yarn exhibits an elasticity higher than an elasticity of the one or more additional yarns and of the second yarn if present; and wherein the textile exhibits an ultimate elongation of about 50% to about 700% and is shape-adapting and biocompatible, is configured to be inserted and/or implanted in a subject's body.

Example 76: The method of any examples herein, particularly example 75, wherein the multi-component yarn is formed by covering the second yarn around the first yarn.

Example 77: The method of any examples herein, particularly examples 75-76, wherein the first polymer comprises a thermoplastic polyurethane (TPU), a polyurethane (PU), a thermoplastic elastomer (TPE), an elastomeric polyolefin, polybutylene terephthalate (PBT), silicone-based elastomer, or a combination thereof.

Example 78: The method of any examples herein, particularly examples 75-77, wherein the first polymer has a durometer from about 75 A to about 75 D.

Example 79: The method of any examples herein, particularly examples 75-78, wherein the first polymer has an ultimate tensile strength between about 4500 psi to about 8000 psi.

Example 80: The method of any examples herein, particularly examples 75-79, wherein the first polymer has an ultimate elongation between about 50% to about 700%.

Example 81: The method of any examples herein, particularly examples 75-80, wherein the first polymer is the TPU.

Example 82: The method of any examples herein, particularly examples 75-81, wherein the first yarn is a monofilament.

Example 83: The method of any examples herein, particularly examples 75-81, wherein the first yarn is a multifilament.

Example 84: The method of any examples herein, particularly example 83, wherein the first yarn has a filament count from greater than about 1 to about 50.

Example 85: The method of any examples herein, particularly examples 75-84, wherein the one or more filaments of the first yarn has a size ranging from about 5 denier to about 1000 denier.

Example 86: The method of any examples herein, particularly examples 75-85, wherein if more than one additional yarns are present, these additional yarns are the same or different.

Example 87: The method of any examples herein, particularly examples 75-86, wherein the one or more additional yarns comprise a fully-drawn yarn, spin-drawn yarn, low- or not-twisted yarn, twisted yarn, a flat yarn, a textured yarn, a high (HS) stretch textured yarn, a high-bulk textured yarn, a high shrinkage yarn, or any combination thereof.

Example 88: The method of any examples herein, particularly examples 75-87, wherein the one or more additional yarns comprise a polyolefin, a polyester, a polyurethane, a thermoplastic polyurethane, a polyamide, or a combination thereof.

Example 89: The method of any examples herein, particularly examples 75-88, wherein the one or more additional yarns are substantially rigid or semi-rigid.

Example 90: The method of any examples herein, particularly examples 75-89, wherein when the second yarn is present, the first polymer has a higher elasticity than the second polymer.

Example 91: The method of any examples herein, particularly examples 75-90, wherein when the second yarn is present, the second polymer comprises a polyester, a nylon, a polyolefin, or a combination thereof.

Example 92: The method of any examples herein, particularly example 91, wherein the second polymer comprises polyethylene terephthalate (PET) homopolymer and copolymers, polypropylene terephthalate (PPT) homopolymer and copolymers and polybutylene terephthalate (PBT) homopolymer and copolymers, or any combination thereof.

Example 93: The method of any examples herein, particularly examples 75-92, wherein when the second yarn is present, the second yarn is a monofilament.

Example 94: The method of any examples herein, particularly examples 75-92, wherein the second yarn is present, the second yarn is a multifilament.

Example 95: The method of any examples herein, particularly example 94, wherein the second yarn has a filament count from greater than 1 to 150.

Example 96: The method of any examples herein, particularly examples 75-95, wherein when the second yarn is present, the one or more filaments of the second yarn has a size ranging from about 5 denier to about 1000 denier.

Example 97: The method of any examples herein, particularly examples 75-96, wherein the multi-component yarn, if present, is single covered or double covered.

Example 98: The method of any examples herein, particularly example 97, wherein the multi-component yarn is double covered, two second yarns cover the first yarn in the opposite direction.

Example 99: The method of any examples herein, particularly examples 75-98, wherein the second yarn comprises a fully-drawn yarn, spin-drawn yarn, low- or not-twisted yarn, twisted yarn, a flat yarn, a textured yarn, a high stretch (HS) textured yarn, a high-bulk textured yarn, a high shrinkage yarn, or any combination thereof.

Example 100: The method of any examples herein, particularly examples 75-99, wherein the one or more filaments of the first yarn is melt-spun.

Example 101: The method of any examples herein, particularly examples 75-100, wherein the step of forming comprises weaving, knitting, or braiding.

Example 102: The method of any examples herein, particularly example 101, wherein the textile is formed by weaving and the second yarn is not present, the first yarn is woven in a weft direction.

Example 103: The method of any examples herein, particularly example 102, wherein the one or more additional yarns are woven in a warp direction.

Example 104: The method of any examples herein, particularly example 102, wherein the one or more additional yarns and the first yarn are woven in a warp direction.

Example 105: The method of any examples herein, particularly example 101, wherein the textile is formed by weaving and the second yarn is present, the multi-component yarn is installed on a first warp-yarn beam, and wherein the one or more additional yarns are installed on a second warp-yarn beam, such that the first warp-yarn bean is under tension different than the tension of the second warp-yarn beam.

Example 106: The method of any examples herein, particularly example 105, wherein the first warp-yarn bean is under higher tension different than the second warp-yarn beam.

Example 107: The method of any examples herein, particularly example 105, wherein the first warp-yarn bean is under lower tension different than the second warp-yarn beam.

Example 108: The method of any examples herein, particularly examples 105-107, wherein the warp direction of the textile comprises the multi-component yarn and the one or more additional yarns in a ratio from about 1:2 to about 1:10.

Example 109: The method of any examples herein, particularly examples 105-108, further comprising a third warp-yarn beam comprising a third yarn that is different from the first yarn.

Example 110: The method of any examples herein, particularly example 109, wherein the third yarn comprises a fully-drawn yarn, spin-drawn yarn, low- or not-twisted yarn, twisted yarn, a flat yarn, a textured yarn, a high (HS) stretch textured yarn, a high-bulk textured yarn, a high shrinkage yarn, or any combination thereof.

Example 111: The method of any examples herein, particularly example 110, wherein the third yarn is the textured yarn.

Example 112: The method of any examples herein, particularly examples 105-111, wherein a weft direction of the textile comprises the one or more additional yarns.

Example 113: The method of any examples herein, particularly examples 109-112, wherein the third yarn is woven to form a longer float or raised feature/s on a surface of the textile when compared to a substantially identical reference textile in the absence of the third yarn.

Example 114: The method of any examples herein, particularly example 113, wherein the textile has a higher surface area when compared to a substantially identical reference textile in the absence of the third yarn.

Example 115: The method of any examples herein, particularly examples 105-114, wherein the textile has a predetermined weave pattern comprising an alternating arrangement of the multi-component yarn side-by-side with the one or more additional yarns in a warp direction.

Example 116: The method of any examples herein, particularly examples 105-115, wherein the one or more additional yarns in the warp direction are the textured yarn, a high (HS) stretch textured yarn, the high-bulk textured yarn and/or a high shrinkage yarn.

Example 117: The method of any examples herein, particularly examples 112-116, wherein the one or more additional yarns in the weft direction are flat or twisted yarns or textured yarns.

Example 118: The method of any examples herein, particularly examples 104-117, wherein a warp density of the multi-component yarn is from about 10 ends/inch to about 200 ends/inch.

Example 119: The method of any examples herein, particularly examples 112-116, wherein a warp density of the textured yarn, the high-bulk textured yarn and/or a high shrinkage yarn is about 10 ends/inch to about 200 ends per inch.

Example 120: The method of any examples herein, particularly examples 112-119, wherein a weft density is from about 10 picks/inch to about 200 picks/inch.

Example 121: The method of any examples herein, particularly examples 101-120, wherein the step of forming is weaving, the textile can a woven construction comprising a plain weave, derivatives of plain weave, a rib weave, a basket weave, a twill weave, a satin weave, a dobby weave, a jacquard weave, an extra yarn weave, a pique weave, a double cloth textile, a crepe weave, a leno weave, a pile weave, a triaxial weave, or a combination thereof.

Example 122: The method of any examples herein, particularly examples 101-121, wherein the textile comprises a simple single jersey knit construction; a single jersey knit with knit-miss pattern construction; a single jersey knit with knit-tuck pattern construction; tricot knit, locknit, satin knit, reveres locknit, sharkskin knit, purl knit, rib stitch knit, interlock stitch knit, double knit, warp knit, raschel knit cable knit, bird's eye knit, pontell knit, intarsia knit, jacquard knit, knitted terry textile, knitted velour textile, sliver knit, fleece knit, or any combination thereof.

Example 123: The method of any examples herein, particularly example 122, wherein the textile is formed by knitting, and wherein the textile does not comprise the second yarn and has a warp-knitted construction, a warp direction of the textile comprises the first yarn and the one or more additional yarns in a ratio from about 1:2 to about 1:10.

Example 124: The method of any examples herein, particularly example 122, wherein the textile is formed by knitting, and wherein the textile has a crochet-knitted construction, a warp direction of the textile comprises the first yarn.

Example 125: The method of any examples herein, particularly example 124, wherein the warp direction further comprises the one or more additional yarns in a ratio from about 1:2 to about 1:10.

Example 126: The method of any examples herein, particularly example 122, wherein the textile is formed by knitting, and wherein the second yarn is present, and the textile has a warp-knitted construction, a warp direction of the textile comprises the multi-component yarn and the one or more additional yarns in a ratio from about 1:2 to about 1:10.

Example 127: The method of any examples herein, particularly examples 123-126, wherein a weft direction of the textile comprises the one or more additional yarns.

Example 128: The method of any examples herein, particularly example 122, wherein the textile is formed by knitting, and wherein the textile does not comprise the second yarn and has a weft-knitted construction, the textile comprises plaiting of the first yarn and the one or more additional yarns.

Example 129: The method of any examples herein, particularly example 101, wherein the step of forming comprising braiding to form a braided construction.

Example 130: The method of any examples herein, particularly example 129, wherein the braided construction comprises a core and a sheath.

Example 131: The method of any examples herein, particularly example 130, wherein if the second yarn is not present, the core of the braided construction comprises the first yarn, and the sheath comprises the one or more additional yarns.

Example 132: The method of any examples herein, particularly example 131, wherein the second yarn is present, the sheath comprises the multi-component yarn.

Example 133: The method of any examples herein, particularly example 132, wherein the braided construction further comprises an additional first yarn that is not covered by the second yarn, wherein the core comprises the additional first yarn.

Example 134: The method of any examples herein, particularly examples 129-133, wherein the braided constructions are biaxial, triaxial, unidirectional, or a combination thereof.

Example 135: The method of any examples herein, particularly examples 75-134, wherein the textile exhibits elastic recovery of at least 80%.

Example 136: The method of any examples herein, particularly examples 75-135, wherein the textile exhibits high stretch, self-conforming to a substrate, a predetermined suture retention, a predetermined cloth density retention, a pore size retention, a thickness retention, or a combination thereof.

Example 137: An implantable prosthetic valve comprising: an annular frame having an inflow end, an outflow end, and a longitudinal axis; and a sealing element secured to the frame, wherein the sealing element comprises the textile of any examples herein, particularly examples 1-68.

Example 138: An implantable prosthetic valve comprising: an annular frame having an inflow end, an outflow end, and a longitudinal axis, wherein the annular frame is configured to expand and collapse; and a sealing element secured to the frame, wherein the sealing element comprises a textile comprising: a) a first yarn comprising one or more filaments comprising a first polymer; b) one or more additional yarns comprising a composition and/or properties different from the first yarn; wherein the first yarn exhibits an elasticity higher than an elasticity of the one or more additional yarns, wherein the textile exhibits an ultimate elongation of about 50% to about 700% and is shape-adapting and biocompatible, and wherein the textile is configured to be inserted and/or implanted in a subject's body.

Example 139: The implantable prosthetic valve of any examples herein, particularly example 138, wherein the textile further comprises a second yarn comprising one or more filaments comprising a second polymer comprising polyester, a nylon, a polyolefin, or a combination thereof, and wherein the second yarn is different from the first yarn, wherein the first polymer has higher elasticity than the second polymer; wherein the second yarn covers the first yarn to form a multi-component yarn such that the first yarn forms a core and the second yarn forms a sheath, wherein the multi-component yarn is single-covered or double-covered, and wherein when the multi-component yarn is double-covered, two second yarns cover the first yarn in an opposite direction.

Example 140: The implantable prosthetic valve of any examples herein, particularly example 138 or 139, wherein the sealing element is an outer skirt configured to establish a seal with a native tissue of a patient.

Example 141: The implantable prosthetic valve of any examples herein, particularly examples 138-140, wherein the sealing element is attached to the annular frame with a fastener.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. Thus, it is understood that the disclosure is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described disclosure or the claims which follow. Therefore, we claim as our disclosure all that comes within the scope and spirit of these claims.

We claim:

1. A textile comprising:
a first yarn comprising one or more filaments comprising a first polymer;
one or more additional yarns comprising a composition and/or properties different from the first yarn;
wherein the first yarn exhibits an elasticity higher than an elasticity of the one or more additional yarns; wherein
the textile exhibits an ultimate elongation of about 50% to about 700% and is shape-adapting and biocompatible, and wherein the textile is configured to be inserted and/or implanted in a subject's body.

2. The textile of claim 1, wherein the first polymer comprises a thermoplastic polyurethane (TPU), a polyurethane (PU), a thermoplastic elastomer (TPE), an elastomeric polyolefin, polybutylene terephthalate (PBT), silicone-based elastomer, or a combination thereof; and/or wherein the first polymer has a durometer from about 75 A to about 75 D and/or wherein the first polymer has an ultimate tensile strength between about 4500 psi to about 8000 psi.

3. The textile of claim 1, wherein the one or more additional yarns comprise a polyolefin, a polyester, a polyurethane, a thermoplastic polyurethane, a polyamide, or a combination thereof.

4. The textile of claim 1 further comprising a second yarn comprising one or more filaments comprising a second polymer comprising polyester, a nylon, a polyolefin, or a combination thereof, and wherein the second yarn is different from the first yarn, wherein the first polymer has higher elasticity than the second polymer, and wherein the one or more filaments of the second yarn has a size ranging from about 5 denier to about 1000 denier.

5. The textile of claim 4, wherein the second yarn covers the first yarn to form a multi-component yarn such that the first yarn forms a core and the second yarn forms a sheath.

6. The textile of claim 5, wherein the multi-component yarn is single-covered.

7. The textile of claim 5 wherein the multi-component yarn is double-covered.

8. The textile of claim 5 wherein when the multi-component yarn is double-covered, and the multi-component yarn comprises two second yarns that cover the first yarn in an opposite direction.

9. The textile of claim 1, wherein the one or more filaments of the first yarn is melt-spun.

10. The textile of claim 1, wherein the textile has a knitted construction and comprises a simple single jersey knit construction.

11. The textile of claim 1 wherein the textile has a woven construction

12. The textile of claim 11 wherein the woven construction comprises a plain weave, derivatives of plain weave, a rib weave, a basket weave, a twill weave, a satin weave, a dobby weave, a jacquard weave, an extra yarn weave, a pique weave, a double cloth textile, a crepe weave, a leno weave, a pile weave, a triaxial weave, or a combination thereof.

13. The textile of claim 1 wherein the textile has a braided construction.

14. The textile of claim 4 wherein the textile is a woven textile, and a warp direction of the woven textile comprises a multi-component yarn and the one or more additional yarns in a ratio from about 1:2 to about 1:10.

15. The textile of claim 4, wherein the textile is a woven textile and the warp direction of the woven textile further comprises a third yarn that is different from the first yarn.

16. The textile of claim 5, wherein the multi-component yarn is woven in the warp direction under a tension different than a tension of the one or more additional yarns woven in the warp direction.

17. The textile of claim 14, wherein the third yarn forms a longer float or raised feature on a surface of the textile when compared to a substantially identical reference textile in the absence of the third yarn.

18. The textile of claim 1, wherein the textile exhibits an elastic recovery of at least 80%.

19. A method comprising:
forming a textile comprising: a first yarn comprising one or more filaments comprising a first polymer;
one or more additional yarns that have a composition and/or properties different from the first yarn; and
optionally a second yarn comprising one or more filaments comprising a second polymer, wherein if the second yarn is present, the second yarn covers the first yarn to form a multi-component yarn such that the first yarn forms a core and the second yarn forms a sheath;
wherein the first yarn exhibits an elasticity higher than an elasticity of the one or more additional yarns and of the second yarn if present; and wherein
the textile exhibits an ultimate elongation of about 50% to about 700% and is shape-adapting and biocompatible, is configured to be inserted and/or implanted in a subject's body.

20. An implantable prosthetic valve comprising:
an annular frame having an inflow end, an outflow end, and a longitudinal axis; wherein the annular frame is configured to expand and collapse; and
a sealing element secured to the frame, wherein the sealing element comprises a textile having a knitted, woven, or braid construction and comprising:
a first yarn comprising one or more filaments comprising a first polymer; and
one or more additional yarns comprising a composition and/or properties different from the first yarn;
wherein the first yarn exhibits an elasticity higher than an elasticity of the one or more additional yarns; and
wherein the textile exhibits an ultimate elongation of about 50% to about 700% and is shape-adapting and biocompatible.
